(19) United States
(12) Patent Application Publication
(10) Pub. No.: US 2023/0139562 A1
(43) Pub. Date: May 4, 2023

(54) MEDICAL IMAGE IMAGING SYSTEM, OPERATION AUTHORITY CONTROL METHOD, AND COMPUTER-READABLE NONVOLATILE STORAGE MEDIUM STORING OPERATION AUTHORITY CONTROL PROGRAM

(71) Applicant: CANON MEDICAL SYSTEMS CORPORATION, Otawara-shi (JP)

(72) Inventors: Sho KAWAJIRI, Nasushiobara (JP); Kensuke SHINODA, Otawara (JP); Kazuyuki SOEJIMA, Otawara (JP); Shinya OZAWA, Nasushiobara (JP); Kazuya TANOUE, Utsunomiya (JP)

(73) Assignee: CANON MEDICAL SYSTEMS CORPORATION, Otawara-shi (JP)

(21) Appl. No.: 18/049,707

(22) Filed: Oct. 26, 2022

(30) Foreign Application Priority Data
Nov. 1, 2021 (JP) 2021-178964

(51) Int. Cl. G16H 40/20; G16H 40/63

(52) U.S. Cl. CPC G16H 40/20; G16H 40/63

(57) ABSTRACT

A medical image imaging system according to an embodiment includes a processing circuitry. The processing circuitry determines a progress in a workflow related to imaging of a subject by a medical image imaging apparatus. The processing circuitry assigns an operation authority for the medical image imaging apparatus to one user terminal among a plurality of user terminals in accordance with the determined progress.

### CROSS-REFERENCE TO RELATED APPLICATIONS

This application is based upon and claims the benefit of priority from Japanese Patent Application No. 2021-178964, filed on Nov. 1, 2021, the entire contents of which are incorporated herein by reference.

### FIELD

Embodiments described herein relate generally to a medical image imaging system, an operation authority control method, and a computer-readable nonvolatile storage medium storing an operation authority control program.

### BACKGROUND

Conventionally, medical information processing systems that provide a mechanism of preventing inadvertent updating of related information about medical images and enabling appropriate operation of the related information have been known. For example, regarding related information of a medical image, if a creator of the related information and an editor of the related information are different, the medical information processing system prohibits the editing.

However, for example, in a case where multiple people operate one modality, while images of a subject are imaged with the modality, information about the imaging may be operated by a user who is not involved in the imaging.

### BRIEF DESCRIPTION OF THE DRAWINGS

FIG. 1 is a diagram illustrating one example of a schematic structure of a medical image imaging system according to an embodiment;

FIG. 2 is a block diagram illustrating a structure example of the medical image imaging system according to the embodiment;

FIG. 3 is a diagram illustrating one example of an examination room in which an imaging unit is installed according to the embodiment;

FIG. 4 is a diagram illustrating one example of an imaging workflow related to an MRI apparatus according to the embodiment;

FIG. 5 is a flowchart expressing one example of a procedure an operation authority control process according to the embodiment; and

FIG. 6 is a diagram illustrating one example of a medical image imaging system according to a modification of the embodiment.

### DETAILED DESCRIPTION

A medical image imaging system according to an embodiment includes a processing circuitry. The processing circuitry determines the progress in the workflow related to the imaging of a subject by a medical image imaging apparatus. The processing circuitry assigns an operation authority for the medical image imaging apparatus to one user terminal among a plurality of user terminals in accordance with the determined progress.

An embodiment of the medical image imaging system, an operation authority control method, and an operation authority control program will be described below with reference to the drawings. In the embodiment below, components denoted with the same reference symbol are assumed to operate in the same way, and the overlapping explanation of such components is omitted as appropriate.

### Embodiment

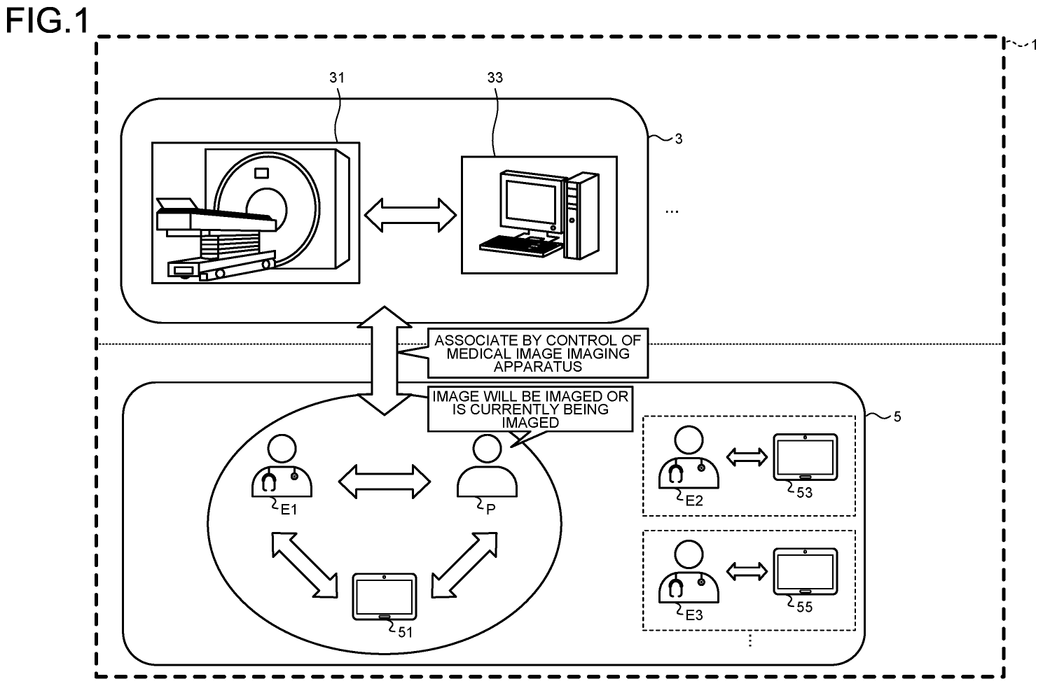

FIG. 1 is a diagram illustrating one example of a schematic structure of a medical image imaging system 1. As illustrated in FIG. 1, the medical image imaging system 1 includes at least one medical image imaging apparatus 3 and a user terminal group 5 that can access the medical image imaging apparatus 3. The medical image imaging apparatus 3 includes an imaging unit 31 capable of capturing images of a subject P, and a console apparatus 33 related to the control of the imaging unit 31. The medical image imaging apparatus 3 corresponds to, for example, various kinds of medical image diagnosis apparatuses including an X-ray computed tomography apparatus (hereinafter referred to as X-ray CT apparatus), an X-ray diagnosis apparatus, a magnetic resonance imaging apparatus (hereinafter referred to as MRI apparatus), a nuclear medicine diagnosis apparatus, and an ultrasonic diagnosis apparatus.

The user terminal group 5 includes a plurality of user terminals (in FIG. 1, first user terminal 51, second user terminal 53, and third user terminal 55) associated with a plurality of users who perform log-in operations (for example, technicians such as, in FIG. 1, first technician E1, second technician E2, and third technician E3). The user terminals are realized, for example, as user interface terminals that can communicate with the medical image imaging apparatus 3. Specifically, the user terminals are realized by wireless hardware such as tablet terminals and smartphones and/or wired terminals carried by in-hospital workers including doctors and technicians, for example. As illustrated in FIG. 1, the first user terminal 51 is associated with the first technician E1 and the subject P. The second user terminal 53 is associated with the second technician E2, and the third user terminal 55 is associated with the third technician E3. To make the description more specific, a tablet terminal is described as the user terminal.

The first technician E1 can perform predetermined operations on the medical image imaging apparatus 3 through the first user terminal 51, the second technician E2 through the second user terminal 53, and the third technician E3 through the third user terminal 55. The predetermined operations are checking and operating about images and patient information related to other subjects who are different from the subject whose image is currently imaged in the medical image imaging apparatus 3. The predetermined operations include, for example, checking the patient information of another subject who is different from the subject whose image is currently being imaged by the medical image imaging apparatus 3, making an appointment of the patient information about the other subject and changing the patient information, checking MR images related to the other subject, post-processes (such as image processing) of the medical images generated by the medical image imaging apparatus 3 regarding the other subject, and transferring the medical image related to the other subject from the medical image imaging apparatus 3 to a server apparatus or the like.

If, for example, an operation authority is assigned to the first user terminal 51 by the assignment of the operation authority to be described below, the first technician E1 can perform various operations related to the operation authority for the medical image imaging apparatus 3 with the first user terminal 51. The various operations related to the operation authority correspond to, for example, at least one of an operation of patient information related to the subject P whose image is being imaged by the medical image imaging apparatus 3, an operation of the patient information related to the subject P whose image is imaged by the medical image imaging apparatus 3, an operation related to start, pause, and stop of the imaging by the medical image imaging apparatus 3, an operation related to control of a couch in the medical image imaging apparatus 3, and the like. Here, the patient information may include, for example, personal information about the subject P, medical interview information, imaging conditions, and images obtained from the imaging of the subject P.

The processes related to the assignment and removal of the operation authority are to be performed by the medical image imaging apparatus 3 of the medical image imaging system 1. To make the description more specific, an MRI apparatus is described as the medical image imaging apparatus 3.

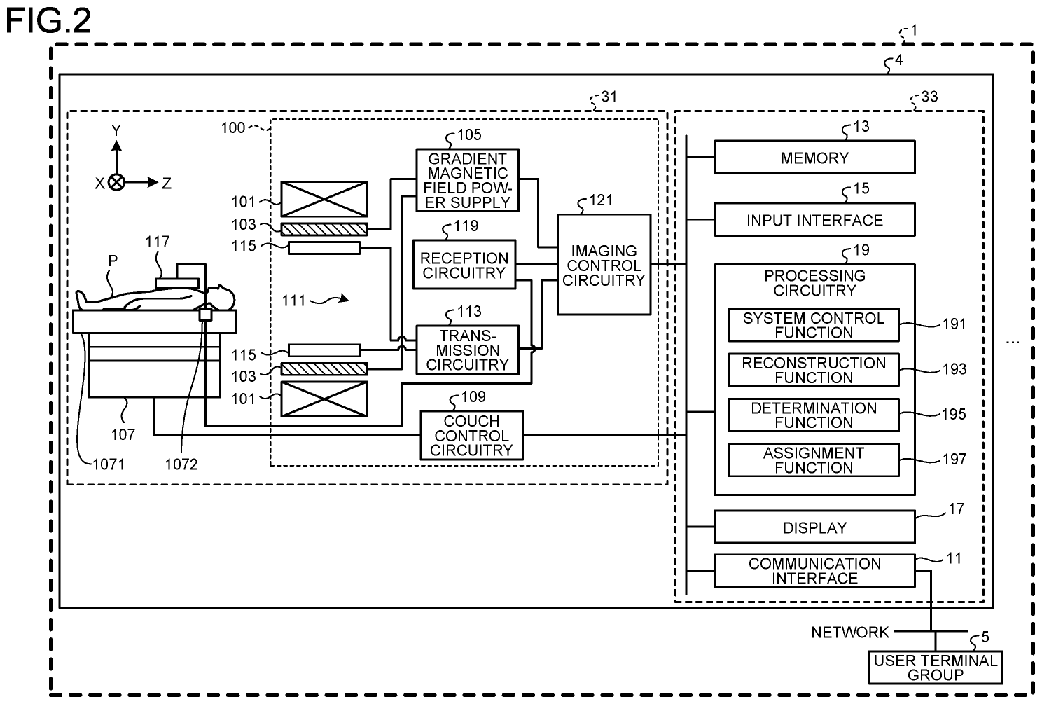

FIG. 2 is a block diagram illustrating a structure example of the medical image imaging system 1 according to the embodiment. As illustrated in FIG. 1, the medical image imaging system 1 includes an MRI apparatus 4 and the user terminal group 5 that is connected to the MRI apparatus 4 in a manner that communication therebetween is possible through a network. The MRI apparatus 4 includes the imaging unit 31 and the console apparatus 33. The imaging unit 31 includes, for example, a gantry 100 and a couch 107. The imaging unit 31 is disposed in an examination room.

The MRI apparatus 4 images the subject P by magnetic resonance imaging. The subject P corresponds to, for example, a patient to be examined with the MRI apparatus 4. The magnetic resonance imaging is an imaging method in which the nuclear spins of the subject P placed in a static magnetic field are magnetically excited by radio frequency (RF) pulses with the Larmor frequency thereof, and images are generated from data of a magnetic resonance signal (hereinafter referred to as MR signal) generated as a result of the excitation. Thus, a strong magnetic field is generated from the MRI apparatus 4.

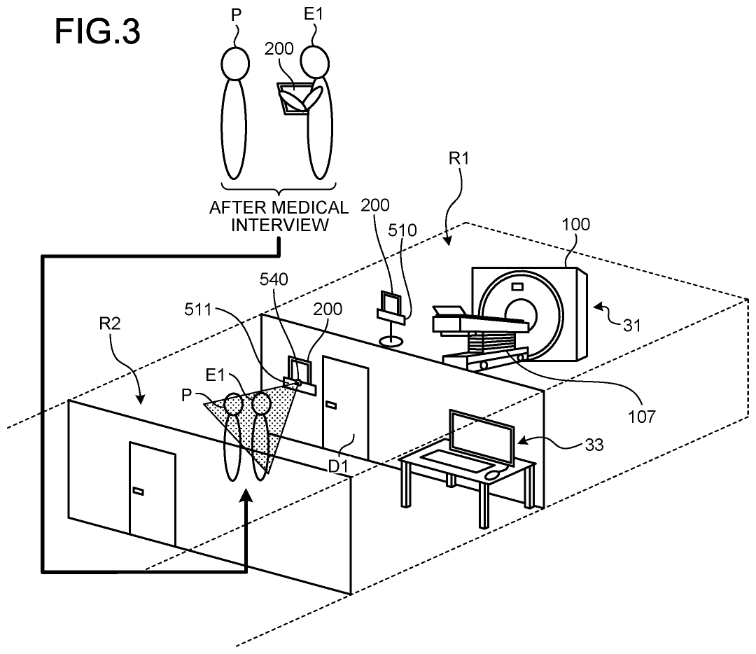

FIG. 3 is a diagram illustrating one example of an examination room R1 where the imaging unit 31 is disposed. The examination room R1 where the imaging unit 31 is installed is a shielded room. The shielded room is realized so that extraneous electromagnetic waves do not enter the examination room R1 and so that the electromagnetic waves generated from the imaging unit 31 are confined within the examination room R1 and do not leak outside.

As illustrated in FIG. 3, the examination room R1 may be equipped with a placement table 510 on which the user terminal can be placed. To make the description more specific, a tablet terminal 200 corresponding to the first user terminal 51 is described as the user terminal illustrated in FIG. 3. The placement table 510 corresponds to a predetermined place where the tablet terminal 200 is placed in the examination room R1. At this time, the first technician E1 may place the tablet terminal 200 on the placement table 510. The placement table 510, for example, functions as a tablet holder. The tablet holder may be provided on the exterior of the gantry 100 on the couch 107 side or on the exterior of the couch 107. A sensor that detects the placement of the tablet terminal 200, such as a contact sensor, is provided on the placement table (hereinafter referred to as in-examination room holder) 510. The contact sensor outputs the detection of the placement of the tablet terminal 200 in the in-examination room holder 510 to the console apparatus 33. As for the sensor that detects the placement of the tablet terminal 200 on the in-examination room holder 510, known technologies can be used as appropriate, and thus the description will be omitted.

A door D1, which is used to enter and exit the examination room R1, is located between the examination room R1 and an anteroom R2 for the examination room R1 (hereinafter referred to as examination anteroom). The subject P and the first technician E1 enter the examination room R1 from the door D1 through the examination anteroom R2. The door D1 also serves as an exit from the examination room R1. The first technician E1 is an example of medical workers. The first technician E1 in the present embodiment may be any other medical worker, such as a doctor or a nurse. The positional relationship between the examination room R1 and the examination anteroom R2 is not limited to the example illustrated in FIG. 3. In the examination anteroom R2, the console apparatus 33 connected to the imaging unit 31 is installed.

The examination anteroom R2 may be equipped with a placement table (hereinafter referred to as in-anteroom holder) 511 on which the tablet terminal 200 can be placed. The in-anteroom holder 511 is installed near the door D1 of the examination anteroom R2, for example, as illustrated in FIG. 3. The in-anteroom holder 511 corresponds to a predetermined place where the tablet terminal 200 is placed in the examination anteroom R2. Before entering the examination room R1 from the examination anteroom R2, the user, for example the first technician E1, places the tablet terminal 200 on the in-anteroom holder 511. The in-anteroom holder 511 includes a sensor, such as a contact sensor, that detects the placement of the tablet terminal 200. The contact sensor outputs the detection of the placement of the tablet terminal 200 on the in-anteroom holder 511 to the console apparatus 33.

An authentication sensor 540 capable of recognizing the user and the subject P is provided on a wall surface of the examination anteroom R2 in the wall that separates the examination room R1 and the examination anteroom R2, for example near the door D1. The authentication sensor 540 corresponds to various sensors used for face authentication, voice authentication, iris authentication, and fingerprint authentication, and the like, for example. The authentication sensor 540 may be installed on a wall surface near the couch 107 or the gantry 100, for example. The output from the authentication sensor 540 is output to the console apparatus 33 through wireless or wired communication. Known technologies can be used as the authentication sensor 540 as appropriate, and thus, the description is omitted.

An optical camera may be provided on the top of the couch 107 and/or on the ceiling in the examination room R1. The optical camera can capture images of the subject P placed on a couchtop 1071 of the couch 107. The optical camera may be disposed near the couch 107 as long as the optical camera can capture images of the subject P placed on the couchtop 1071. The output from the optical camera is output to the console apparatus 33 through wireless or wired communication.

The tablet terminal 200 includes, for example, a storage circuitry, a processing circuitry such as a CPU, a network interface, an input interface, a display, a camera, and the like. The input interface of the tablet terminal 200 is, for example, a touch screen in which a display and a touch pad are unified.

The tablet terminal 200 is used, for example, by the first technician E1 to interview the subject P before the subject P is examined with the MRI apparatus 4. Various kinds of information are input into the tablet terminal 200, for example, through the interview between the first technician E1 who operates the MRI apparatus 4 and the subject P who is to be examined with the MRI apparatus 4. The tablet terminal 200 has a wireless communication function and connects to the console apparatus 33 of the MRI apparatus 4 in a manner that communication therebetween is possible. The tablet terminal 200 may be connected to information processing systems within a medical institution, such as a hospital information system (HIS) or a radiology information system (RIS), in a manner that communication therebetween is possible.

As illustrated in FIG. 2, the MRI apparatus 4 and the tablet terminal 200 (included in user terminal group 5 in FIG. 2) are connected by a network. The network is, for example, an in-hospital local area network (LAN). The network may further be connected to HIS and RIS including electronic medical record systems, various server apparatuses, or the like installed within the medical institution.

As illustrated in FIG. 2, the MRI apparatus 4 includes the gantry 100, the couch 107, and the console apparatus 33. The gantry 100 includes an image imaging system related to the imaging of the subject P to be examined with the MRI apparatus 4. The exterior of the gantry 100 may include the authentication sensor, the optical camera, and an operation panel for the movement of the couch 107 or the couchtop 1071, for example. Specifically, the gantry 100 includes a static magnetic field magnet 101, a static magnetic field power supply (not illustrated), a gradient coil 103, a gradient magnetic field power supply 105, a couch control circuitry 109 that controls the couch 107, a transmission coil 115, a transmission circuitry 113, a reception circuitry 119, and an imaging control circuitry 121. The gantry 10 may include a reception coil 117.

The structure illustrated in FIG. 2 is just one example. At least one of the static magnetic field power supply (not illustrated), the gradient magnetic field power supply 105, the couch control circuitry 109, the transmission circuitry 113, and the reception circuitry 119 may be disposed in, for example, the examination room R1, the examination anteroom R2, or the like instead of being disposed on the gantry 100. The MRI apparatus 4 does not include the subject P.

The static magnetic field magnet 101 is a hollow magnet with an approximately cylindrical shape. The static magnetic field magnet 101 generates an approximately uniform static magnetic field in the interior space. For example, a superconducting magnet or the like is used as the static magnetic field magnet 101.

The gradient coil 103 is a hollow coil with an approximately cylindrical shape, and is placed on the inner surface side of a cylindrical cooling vessel. The gradient coil 103, upon the reception of an individual current supply from the gradient magnetic field power supply 105, generates a gradient magnetic field whose magnetic field strength varies along the mutually orthogonal X, Y, and Z axes. The gradient magnetic fields in the X, Y, and Z axes generated by the gradient coil 103 form, for example, a gradient magnetic field for slice selection, a gradient magnetic field for phase encoding, and a gradient magnetic field for frequency encoding (also called readout gradient magnetic field). The gradient magnetic field for slice selection is used to arbitrarily determine the imaging cross section. The gradient magnetic field for phase encoding is used to change the phase of the magnetic resonance signal (hereafter referred to as MR signal) according to a spatial position. The gradient magnetic field for frequency encoding is used to change the frequency of the MR signal according to the spatial position.

The gradient magnetic field power supply 105 is a power supply apparatus that supplies current to the gradient coil 103 under the control of the imaging control circuitry 121.

The couch 107 is a apparatus including the couchtop 1071 on which the subject P is placed. Under the control by the couch control circuitry 109, the couch 107 inserts the couchtop 1071 on which the subject P is placed into a bore (opening of the gantry 100) 111. The exterior of the couch 107 may include the authentication sensor 540, the optical camera, and an operation panel for the movement of the couch 107 or the couchtop 1071, for example. At least one coil port 1072 is installed at four corners of the couchtop 1071 and/or at an end of the couchtop 1071 in a short-axis direction thereof. The coil port 1072 is connected to a connection terminal of a local transmission/reception RF coil and/or the reception coil 117 according to an imaging site of the subject P. At this time, the couch 107 outputs to the console apparatus 33, the presence or absence of a connection between the connection terminal of the transmission/reception RF coil and/or the reception coil 117, and the coil port 1072.

The couch control circuitry 109 is a circuitry that controls the couch 107. The couch control circuitry 109 drives the couch 107 by the operator's instruction through an input interface 15 to move the couchtop 1071 in a longitudinal direction and in an up-down direction, and in some cases, in a left-right direction.

The transmission circuitry 113 supplies high-frequency pulses modulated at the Larmor frequency to the transmission coil 115 under the control of the imaging control circuitry 121. For example, the transmission circuitry 113 includes an oscillation unit, a phase selection unit, a frequency conversion unit, an amplitude modulation unit, an RF amplifier, and the like. The oscillation unit generates RF pulses with the resonance frequency unique to the target nucleus in a static magnetic field. The phase selection unit selects the phase of the RF pulses generated by the oscillation unit. The frequency conversion unit converts the frequency of the RF pulses output from the phase selection unit. The amplitude modulation unit modulates the amplitude of the RF pulses output from the frequency conversion unit according to a sinc function, for example. The RF amplifier amplifies the RF pulses output from the amplitude modulation unit and supplies the amplified RF pulses to the transmission coil 115.

The transmission coil 115 is an RF coil disposed inside the gradient coil 103. The transmission coil 115 generates RF pulses corresponding to a high-frequency magnetic field in response to the output from the transmission circuitry 113.

The reception coil 117 is an RF coil disposed inside the gradient coil 103. The reception coil 117 receives the MR signals emitted from the subject P by the high-frequency magnetic field. The reception coil 117 outputs the received MR signals to the reception circuitry 119. The reception coil 117 is, for example, a coil array with one or more, typically multiple, coil elements. The reception coil 117 may be formed by one coil element. Although the transmission coil 115 and the reception coil 117 are described as separate RF coils in FIG. 2, the transmission coil 115 and the reception coil 117 may be implemented as an integrated transmission/reception coil. The transmission/reception coil corresponds to the imaging site of the subject P, and is, for example, a local transmission/reception RF coil such as a head coil. When the local transmission/reception RF coil is used for the imaging, the transmission/reception RF coil or the reception coil 117 is connected to the coil port 1072 in the couchtop 1071 by the first technician E1.

The reception circuitry 119 generates digital MR signals (hereinafter referred to as MR data) on the basis of the MR signals output from the reception coil 117 under the control of the imaging control circuitry 121. Specifically, the reception circuitry 119 performs various signal processing on the MR signals output from the reception coil 117, and then performs analog-to-digital (A/D) conversion on the data resulting from the various signal processing to generate the MR data. The reception circuitry 119 outputs the generated MR data to the imaging control circuitry 121. For example, the MR data is generated for each coil element and output to the imaging control circuitry 121 along with a tag identifying the coil element.

The imaging control circuitry 121 controls the gradient magnetic field power supply 105, the transmission circuitry 113, the reception circuitry 119, and the like according to an imaging protocol output from a processing circuitry 19 to perform the imaging of the subject P. The imaging protocol has a pulse sequence for each type of examination. In the imaging protocol, the amount of current supplied to the gradient coil 103 by the gradient magnetic field power supply 105, a timing when the current is supplied to the gradient coil 103 by the gradient magnetic field power supply 105, the magnitude or time width of the high-frequency pulses supplied to the transmission coil 115 by the transmission circuitry 113, a timing when the high-frequency pulses are supplied to the transmission coil 115 by the transmission circuitry 113, a timing when the MR signal is received by the reception coil 117, and the like are defined.

The imaging control circuitry 121 drives the gradient magnetic field power supply 105, the transmission circuitry 113, the reception circuitry 119, and the like to execute the imaging of the subject P, and upon the reception of the resulting MR data from the reception circuitry 119, the imaging control circuitry 121 transfers the received MR data to the console apparatus 33 and the like. The imaging control circuitry 121 is realized by, for example, a processor.

Although “the processor” reads out a computer program corresponding to each function from a memory 13 and executes the computer program in the above example, the embodiment is not limited to this. The term “processor” includes, for example, a CPU, a graphics processing unit (GPU), an application specific integrated circuit (ASIC), a programmable logic device (e.g., simple programmable logic device (SPLD)), a complex programmable logic device (CPLD), a field programmable gate array (FPGA), and other circuits.

If the processor is, for example, a CPU, the processor reads out and executes a computer program saved in the memory 13 to realize the function. On the other hand, if the processor is an ASIC, the function is incorporated directly into a circuit of the processor as a logic circuit instead of saving the computer program in the memory 13. Each processor in this embodiment is not limited to the processor configured as a single circuit for each processor, but may alternatively be configured as a single processor by combining a plurality of independent circuits to realize the function. Although a single storage circuit is described as storing the computer program corresponding to each processing function, a plurality of storage circuits may be distributed and the processing circuit may read out the corresponding computer program from the individual storage circuit.

The console apparatus 33 controls the entire MRI apparatus 4 and generates the MR images, for example. As illustrated in FIG. 3, the console apparatus 33 is installed in the examination anteroom R2, an operation room, or the like. The functions of the processing circuitry 19 in the console apparatus 33 that perform various processes may be installed in a separate calculation room as a calculation apparatus. As illustrated in FIG. 2, the console apparatus 33 includes a communication interface 11, the memory 13, the input interface 15, a display 17, the processing circuitry 19, and the like.

The communication interface 11 performs data communication with, for example, the user terminal group 5, HIS, RIS, PACS, and the like. The standard for communication between the communication interface 11 and the user terminal group 5, HIS, RIS, PACS, etc. may be any standard, such as Health Level 7 (HL7), Digital Imaging and Communications in Medicine (DICOM), or both. The communication interface 11 corresponds to a communication unit.

The memory 13 stores therein various kinds of information received by the communication interface 11, k-space data placed in k-space by the processing circuitry 19, which is described below, image data generated by the processing circuitry 19, and the like. The memory 13 stores therein various kinds of functions realized by the processing circuitry 19, such as a system control function 191, a reconstruction function 193, a determination function 195, and an assignment function 197, as computer-executable computer programs.

The memory 13 is realized by, for example, a semiconductor memory element such as a random access memory (RAM) or a flash memory, a hard disk drive (HDD), a solid state drive (SSD), an optical disk, or the like. The memory 13 may be realized by a compact disc (CD)-ROM drive, a digital versatile disc (DVD) drive, or a driving apparatus that reads and writes various information from and to a portable storage medium such as a flash memory, for example. The memory 13 corresponds to a storage unit.

The input interface 15 receives various instructions and information input from the operator. The input interface 15 is formed by, for example, a trackball, a switch button, a mouse, a keyboard, a touchpad that receives the input operation by a touch on an operation surface, a touchscreen that integrates a display screen and a touchpad, a non-contact input circuitry using an optical sensor, a voice input circuitry, or the like. The input interface 15 is connected to the processing circuitry 19, converts the input operation received from the operator into electrical signals, and outputs the electric signals to the processing circuitry 19.

The input interface 15 herein is not limited to only those with physical operating components such as a mouse and a keyboard. For example, a processing circuitry for electrical signals that receives electrical signals corresponding to input operations from an external input apparatus installed separately from the console apparatus 33 and outputs these electrical signals to a control circuitry is also one example of the input interface 15. The input interface 15 is realized by a user terminal, such as the tablet terminal 200, for example.

The display 17 displays various graphical user Interfaces (GUIs), guidance screens for the first technician E1, magnetic resonance images generated by the processing circuitry 19, and the like under the control of the processing circuitry 19. The display 17 is a display apparatus, such as a liquid crystal display.

The processing circuitry 19 controls the entire MRI apparatus 4. More specifically, the processing circuitry 19 has, as an example, the system control function 191, the reconstruction function 193, the determination function 195, and the assignment function 197. The processing circuitry 19, which realizes the system control function 191, the reconstruction function 193, the determination function 195, and the assignment function 197, corresponds to a system control unit, a reconstruction unit, a determination unit, and an assignment unit. Each of the functions including the system control function 191, the reconstruction function 193, the determination function 195, the assignment function 197, and the like is stored in the memory 13 as a computer-executable computer program. The processing circuitry 19 is a processor. For example, the processing circuitry 19 reads out a computer program from the memory 13 and executes the computer program to realize the function corresponding to the computer program. In other words, the processing circuitry 19 that has read out the computer program has each of the following functions: the system control function 191, the reconstruction function 193, the determination function 195, the assignment function 197, and the like.

The processing circuitry 19 controls the MRI apparatus 4 with the system control function 191. Specifically, the system control function 191 reads out a system control program stored in the memory 13, develops the read program on the memory, and controls each circuitry of this MRI apparatus 4 according to the developed system control program. For example, the system control function 191 reads out the imaging protocol from the memory 13 on the basis of the imaging condition input by the operator through the input interface 15. The system control function 191 transmits the imaging protocol to the imaging control circuitry 121 to control the imaging of the subject P.

The processing circuitry 19 causes the reconstruction function 193 to arrange the MR data generated by the reception circuitry 119 in the k-space. The reconstruction function 193 performs Fourier transform on the MR data arranged in the k-space to generate a magnetic resonance image. The reconstruction function 193 saves the generated magnetic resonance images in the memory 13, for example.

The processing circuitry 19 causes the determination function 195 to determine the progress in the workflow related to the imaging of the subject P by the MRI apparatus 4 (hereinafter referred to as the imaging workflow). Specifically, the determination function 195 determines the progress in the imaging workflow related to the assignment of the operation authority on the basis of the output from various detectors, such as a contact sensor and the authentication sensor 540, the optical camera, and the like. For example, based on the outputs from various detectors, such as the contact sensor and the authentication sensor 540, the optical camera, and the like, the determination function 195 determines the attainment to the stage before the imaging of the subject P (hereinafter referred to as pre-imaging stage) to be the progress related to the assignment of the operation authority to the tablet terminal 200. The progress related to the assignment of the operation authority corresponding to the pre-imaging stage includes, for example, at least one of a timing when the subject P is guided to the examination room R1 where the MRI apparatus 4 is installed (hereinafter referred to as room entry guidance timing), a timing when the subject P is placed on the couchtop 1071 of the couch 107 in the MRI apparatus 4 (hereinafter referred to as placement timing), a timing when the couchtop 1071 is moved to the opening 111 of the gantry 100 including the image imaging system in the MRI apparatus 4 (hereinafter referred to as movement timing), and a timing when the imaging condition related to the imaging is set in the examination room R1 (hereinafter referred to as setting timing). The room entry guidance timing, the placement timing, the movement timing, and the setting timing will be described in detail below.

The processing circuitry 19 causes the determination function 195 to determine a timing after the imaging of the subject P to be the progress in the imaging workflow. For example, in the progress in the imaging workflow, the determination function 195 determines the attainment to the stage after the imaging of the subject P (hereinafter referred to as post-imaging stage) to be the progress in the imaging workflow to remove the operation authority from the tablet terminal 200 to which the operation authority has been assigned. The post-imaging stage is, for example, the timing when the subject P is guided from the examination room R1 where the MRI apparatus 4 is installed to the outside the examination room R1 (hereinafter referred to as room exit guidance timing).

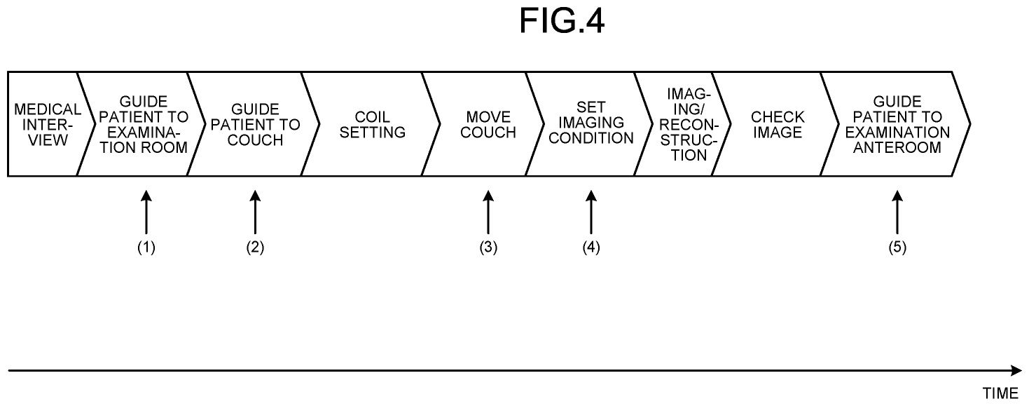

FIG. 4 is a diagram illustrating an example of the imaging workflow related to the MRI apparatus 4. The imaging workflow is different depending on the type of medical image imaging apparatus 3 in the medical image imaging system 1. For example, if the medical image imaging apparatus 3 is an X-ray CT apparatus, “coil setting” illustrated in FIG. 4 is unnecessary. Guiding the patient to the examination room R1 in FIG. 4 includes the room entry guidance timing (1). Guiding the patient to the couch 107 in FIG. 4 corresponds to the placement timing (2). Moving the couch in FIG. 4 corresponds to the movement timing (3). Setting the imaging condition in FIG. 4 corresponds to the setting timing (4). In other words, in the imaging workflow illustrated in FIG. 4, the pre-imaging stage includes the stage of guiding the patient to the examination room R1, the stage of guiding the patient to the couch 107, the stage of moving the couch, and the stage of setting the imaging condition.

In the imaging workflow in FIG. 4, guiding the patient to the examination anteroom R2 includes the room exit guidance timing (5). In other words, guiding the patient to the examination anteroom R2 corresponds to the post-imaging stage in the imaging workflow illustrated in FIG. 4. The room entry guidance timing (1), the placement timing (2), the movement timing (3), the setting timing (4), and the room exit guidance timing (5) are described below.

(1) Room Entry Guidance Timing

The room entry guidance timing is at least one of a timing when one user terminal approaches within the range of a predetermined distance from the examination room R1, a timing when one user terminal is placed at a predetermined place before the examination room R1, and a timing when the subject P or a user who operates one tablet terminal 200 is authenticated about the entry into the examination room R1. The predetermined distance is, for example, a distance at which the tablet terminal 200 as a user terminal and the console apparatus 33 are capable of short-range communication, and corresponds to a distance of several meters from the console apparatus 33, for example.

Specifically, the communication interface 11 receives from RIS, the patient's appointment information related to the imaging in the MRI apparatus 4 (hereinafter referred to as patient appointment information). When the tablet terminal 200 approaches within the range of the predetermined distance from the examination room R1, the communication interface 11 receives the interview result of the subject P from the tablet terminal 200. The processing circuitry 19 causes the determination function 195 to look for the patient appointment information that matches the patient information in the interview result. If the patient information in the interview result matches the next imaging in the patient appointment information (the earliest examination at the present moment), the determination function 195 determines the progress in the imaging workflow to be the pre-imaging stage.

When one tablet terminal 200 is placed at a predetermined position in the examination anteroom R2, the determination function 195 determines the progress in the imaging workflow to be the pre-imaging stage. Specifically, when the tablet terminal 200 is placed in the in-anteroom holder 511 in the examination anteroom R2 by the first technician E1, the contact sensor detects the placement of the tablet terminal 200 in the in-anteroom holder 511 and outputs a signal related to the detection of the placement of the tablet terminal 200 (hereinafter called detection signal) to the processing circuitry 19. Upon the reception of the detection signal, the processing circuitry 19 causes the determination function 195 to determine the progress in the imaging workflow to be the pre-imaging stage.

If the subject P or the first technician E1 who operates one tablet terminal 200 is authenticated about entering the examination room R1, the determination function 195 determines the progress in the imaging workflow to be the pre-imaging stage. Specifically, when at least one of the first technician E1 and the subject P is authenticated by the authentication sensor 540 in the examination anteroom R2, the authentication sensor 540 outputs a signal related to the authentication of at least one of the first technician E1 and the subject P (hereinafter referred to as an authentication signal) to the processing circuitry 19. Upon the reception of the authentication signal, the processing circuitry 19 causes the determination function 195 to determine the progress in the imaging workflow to be the pre-imaging stage.

(2) Placement Timing

The placement timing is at least one of a timing when one user terminal approaches within the range of a first distance from the couch 107, a timing when one user terminal is placed at a predetermined place in the examination room R1, a timing when the subject P or the first technician E1 who operates one user terminal is authenticated within the range of a second distance from the couch 107 or the gantry 100, and a timing when the first technician E1 touches an operation panel for movement of the couch 107 or the couchtop 1071. The first distance is the distance at which the tablet terminal 200 and the gantry 100 are capable of short-range communication in the examination room R1 and corresponds to several meters from the gantry 100, for example. The second distance is the distance at which the first technician E1 or the subject P can be authenticated by the authentication sensor on the gantry 100 and/or the couch 107 in the examination room R1 and corresponds to several tens of centimeters from the authentication sensor, for example.

Specifically, when one tablet terminal 200 approaches within the range of the first distance from the couch 107, the communication interface 11 receives the interview result of the subject P from the tablet terminal 200. The processing circuitry 19 causes the determination function 195 to look for the patient appointment information that matches the patient information in the interview result. If the patient information in the interview result matches the next imaging in the patient appointment information (the earliest examination at the present moment), the determination function 195 determines the progress in the imaging workflow to be the pre-imaging stage.

When one tablet terminal 200 is placed at a predetermined place in the examination room R1, the processing circuitry 19 causes the determination function 195 to determine the progress in the imaging workflow to be the pre-imaging stage. Specifically, when the tablet terminal 200 is placed in the in-examination room holder 510 in the examination room R1, the contact sensor detects the placement of the tablet terminal 200 in the in-examination room holder 510 and outputs the detection signal to the processing circuitry 19. Upon the reception of the detection signal, the processing circuitry 19 causes the determination function 195 to determine the progress in the imaging workflow to be the pre-imaging stage.

If the subject P or the first technician E1 who operates one tablet terminal 200 is authenticated within the range of the second distance from the couch 107 or the gantry 100, the processing circuitry 19 causes the determination function 195 to determine the progress in the imaging workflow to be the pre-imaging stage. Specifically, when at least one of the first technician E1 and the subject P is authenticated by the authentication sensor installed in the couch 107 and/or the gantry 100, the authentication sensor outputs the authentication signal to the processing circuitry 19. Upon the reception of the authentication signal, the determination function 195 determines the progress in the imaging workflow to be the pre-imaging stage.

When the first technician E1 touches the operation panel for the movement of the couch 107 or the couchtop 1071, the processing circuitry 19 causes the determination function 195 to determine the progress in the imaging workflow to be the pre-imaging stage. Specifically, when the first technician E1 touches the operation panel, the contact sensor on the operation panel detects the contact of the first technician E1 on the operation panel and outputs the detection signal to the processing circuitry 19. Upon the reception of the detection signal, the determination function 195 determines the progress in the imaging workflow to be the pre-imaging stage.

(3) Movement Timing

The movement timing is at least one of a timing when the first technician E1 inputs an operation of the couchtop 1071 on an operation panel for movement of the couchtop 1071, a timing when movement control of the couchtop 1071 is input by one user terminal, and a timing when the subject P placed on the couchtop 1071 is specified based on output from the optical camera.

Specifically, when the first technician E1 inputs the operation of the couchtop 1071 on the operation panel for the movement of the couch 107 or the couchtop 1071, the processing circuitry 19 causes the determination function 195 to determine the progress in the imaging workflow to be the pre-imaging stage. More precisely, when the first technician E1 inputs the operation of the couchtop 1071 on the operation panel, the operation panel detects the user's input on the operation panel and outputs a signal related to the detection of the input (hereinafter referred to as panel input detection signal) to the processing circuitry 19. Upon the reception of the panel input detection signal, the determination function 195 determines the progress in the imaging workflow to be the pre-imaging stage.

When the movement control of the couchtop 1071 is input with one tablet terminal 200, the processing circuitry 19 causes the determination function 195 to determine the progress in the imaging workflow to be the pre-imaging stage. Specifically, when the movement control of the couchtop 1071 is input by the first technician E1 who has entered the examination room R1 through the tablet terminal 200 related to the first technician E1, the communication interface 11 receives the control signal related to the movement control of the couchtop 1071 from the tablet terminal 200. The communication interface 11 outputs the received control signal to the processing circuitry 19. Upon the reception of the control signal, the determination function 195 determines the progress in the imaging workflow to be the pre-imaging stage.

The optical camera outputs images related to the couchtop 1071 to the processing circuitry 19. The processing circuitry 19 causes the determination function 195 to specify the subject P placed on the couchtop 1071 on the basis of the image output from the optical camera. Since the subject P can be specified by known image recognition techniques, the description is omitted. Upon specifying the subject P in the output (image) from the optical camera, the determination function 195 determines the progress in the imaging workflow to be the pre-imaging stage.

(4) Setting Timing

The setting timing is at least one of a timing when the imaging condition is set by one user terminal in the examination room R1, a timing when the imaging condition is set by one user terminal in a state where the subject P is moved to the center of the imaging in the opening 111, and a timing when the first technician E1 exits from the examination room R1 in the state where the subject P is moved to the center of the imaging. Note that the setting timing may be a timing when the connection terminal of the reception coil 117 is connected to the coil port 1072. The center of the imaging specifically corresponds to the center of the magnetic field at the opening 111.

Specifically, in response to the input of the imaging condition by the tablet terminal 200 in the examination room R1, or the input of the imaging condition by the tablet terminal 200 in a state where the subject P is moved to the center of the magnetic field at the opening 111 (state where the setting of the subject P regarding the imaging is completed), the communication interface 11 receives the input imaging condition and outputs the received imaging condition to the processing circuitry 19. Upon the reception of the imaging condition, the processing circuitry 19 causes the determination function 195 to determine the progress in the imaging workflow to be the pre-imaging stage.

Upon the exit of the first technician E1 from the examination room R1 in the state where the subject P is moved to the center of the magnetic field (state where the setting of the subject P regarding the imaging is completed), the processing circuitry 19 causes the determination function 195 to determine the progress in the imaging workflow to be the pre-imaging stage. The exit of the user from the examination room R1 is detected by known techniques including, for example, the detection of opening/closing of the door D1, the recognition of the first technician E1 by the optical camera in the examination anteroom R2, the detection of the distance from the gantry 100 to the tablet terminal 200, and the like; thus, the description is omitted.

(5) Room Exit Guidance Timing

The room exit guidance timing is at least one of a timing when the couchtop 1071 of the couch 107 in the MRI apparatus 4 is separated from the center of the imaging of the opening 111 of the gantry 100 including the image imaging system in the MRI apparatus 4, a timing when the couchtop 1071 descends toward a floor surface of the examination room R1, a timing when the reception coil 117 is detached from the coil port 1072 at the couchtop 1071, a timing when separation of the subject P from the couchtop 1071 is specified based on output from the optical camera, a timing when one user terminal has reached a third distance from the gantry 100 or the couch 107, and a timing when end of the imaging is input in one user terminal. The third distance is the distance at which the tablet terminal 200 and the console apparatus 33 become incapable of short-range communication and corresponds to more than several meters, for example.

Specifically, when at least one of the movement of the couchtop 1071 from the center of the magnetic field and the descent of the couchtop 1071 toward the floor surface is performed by the couch control circuitry 109, the processing circuitry 19 causes the determination function 195 to determine the progress in the imaging workflow to be the post-imaging stage. When the coil port 1072 and the connection terminal of the reception coil 117 are disconnected, the determination function 195 determines the progress in the imaging workflow to be the post-imaging stage.

Upon specifying the separation of the subject P from the couchtop 1071 in the image output from the optical camera, the processing circuitry 19 causes the determination function 195 to determine the progress in the imaging workflow to be the post-imaging stage. The separation of the subject P from the couchtop 1071 in the image can be specified based on the image recognition result in which another subject except the couchtop 1071 and the subject P exists between the couchtop 1071 and the subject P in the image, for example. Since the specification is possible by the known image recognition techniques, the description is omitted.

When one tablet terminal 200 has reached the third distance from the gantry 100 or the couch 107, the processing circuitry 19 causes the determination function 195 to determine the progress in the imaging workflow to be the post-imaging stage. For example, the determination function 195 determines the progress in the imaging workflow to be the post-imaging stage when the short-range communication between the tablet terminal 200 and the console apparatus 33 has stopped.

The processing circuitry 19 causes the determination function 195 to determine the progress in the imaging workflow to be the post-imaging stage when the end of imaging of the subject P is input at the tablet terminal 200 for which the operation authority has been set. Setting the operation authority corresponds to, for example, lifting restrictions on various operations of the MRI apparatus 4.

The processing circuitry 19 causes the assignment function 197 to assign the operation authority for the MRI apparatus 4 to one of the user terminals in accordance with the progress determined by the determination function 195. Specifically, in response to the determination that the progress in the imaging workflow has attained the pre-imaging stage, the assignment function 197 sets the operation authority for the MRI apparatus 4 to one of the user terminals that is related to the determination about attaining the pre-imaging stage. In addition, when the determination unit has determined the timing after the imaging of the subject to be the progress in the workflow, the assignment function 197 removes the operation authority assigned to one user terminal. In other words, the assignment function 197 provides the operation authority to the tablet terminal 200 that is determined to be in the pre-imaging stage in the progress in the imaging workflow, and removes the provided operation authority from the tablet terminal 200 that is determined to be in the post-imaging stage in the progress in the imaging workflow.

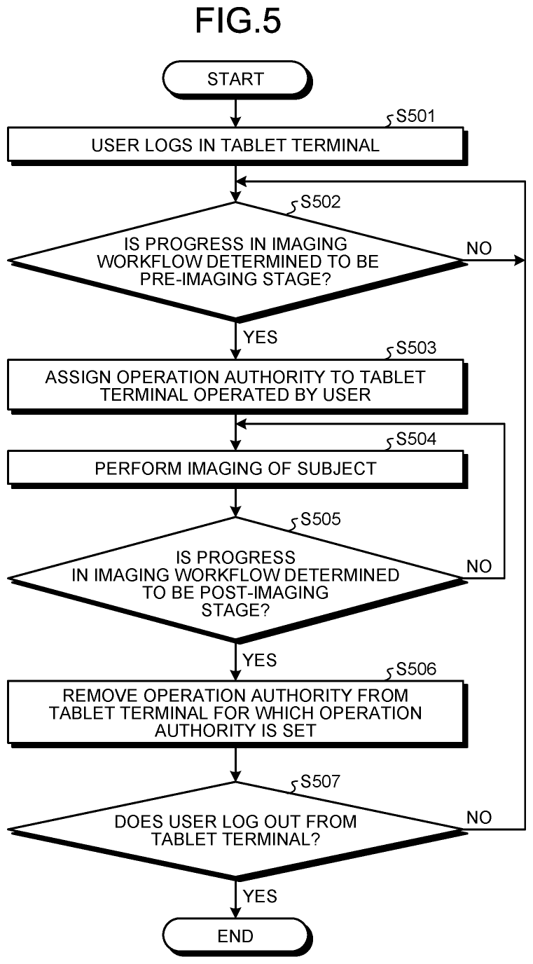

The processes related to the assignment and removal of the operation authority (hereinafter referred to as operation authority control process) to be performed by the medical image imaging system 1 in this embodiment configured as described above are described with reference to FIG. 5. FIG. 5 is a flowchart expressing one example of the procedure of the operation authority control process according to the present embodiment.

Operation Authority Control Process

Step S501

The first technician E1 logs in the tablet terminal 200, which corresponds to the first user terminal 51. The login to the tablet terminal 200 is performed by the authentication sensor in the tablet terminal 200. For example, the login can be achieved through, for example, fingerprint authentication, face authentication, voice authentication, iris authentication, or the like. Thus, the first user terminal 51 (tablet terminal 200), the subject P, and the first technician E1 are associated with each other as illustrated in FIG. 1. The logged-in tablet terminal 200 can perform various operations with the instructions of the first technician E1.

Step S502

If the determination function 195 determines the progress in the imaging workflow to be the pre-imaging stage (Yes at step S502), the process at step 5503 is performed. If the determination function 195 does not determine the progress in the imaging workflow to be the pre-imaging stage (No at step S502), this step is repeated. At this time, if the first technician E1 inputs logout from the tablet terminal 200, the operation authority control process ends.

Step S503

The processing circuitry 19 causes the assignment function 197 to assign the operation authority for the MRI apparatus 4 to the tablet terminal 200 whose progress in the imaging workflow has been determined to be the pre-imaging stage. Restrictions on various operations related to the MRI apparatus 4 are lifted from the tablet terminal 200 for which the operation authority has been set. At this time, the first technician E1 can perform various operations on the MRI apparatus 4 through the tablet terminal 200 without restrictions.

Step S504

The imaging is performed on the subject P according to the instructions of the first technician E1 through the tablet terminal 200 or the console apparatus 33. For example, in response to pressing a button to start the imaging on the tablet terminal 200, the system control function 191 controls the imaging control circuitry 121 to perform the imaging of the subject P.

Step S505

If the determination function 195 determines the progress in the imaging workflow to be the post-imaging stage (Yes at step S505), the process at step S506 is performed. If the determination function 195 does not determine the progress in the imaging workflow to be the post-imaging stage (No at step S505), the process at step S504 is repeated.

Step S506

The processing circuitry 19 causes the assignment function 197 to remove the operation authority from the tablet terminal 200 for which operation authority has been set. Thus, the operation of the MRI apparatus 4 by the tablet terminal 200 is restricted.

Step S507

If the first technician E1 logs out from the tablet terminal 200 (Yes at step S507), the operation authority control process ends. If the first technician E1 does not log out from the tablet terminal 200 (No at step S507), the processes at and after step S502 are repeated.

In the medical image imaging system 1 according to the aforementioned embodiment, the progress in the imaging workflow related to the imaging of the subject P by the medical image imaging apparatus 3 is determined, and based on the progress determined by the determination function 195, the operation authority for the medical image imaging apparatus 3 is assigned to one user terminal among the user terminals. In addition, this medical image imaging system 1 removes the operation authority assigned to one user terminal when the determination function 195 has determined the timing after imaging of the subject P to be the progress in the imaging workflow.

Thus, by the medical image imaging system 1 according to the embodiment, the switching of the assignment and removal of the operation authority can be performed automatically at the appropriate timing in accordance with the imaging workflow as a trigger. In other words, by the present medical image imaging system 1, the MRI apparatus 4 can be controlled without restrictions by the user terminal to which the operation authority has been assigned. Thus, because of having the function of restricting the operation of the patient information during the imaging to the particular first technician E1 or the tablet terminal 200 corresponding to the first user terminal 51, the present medical image imaging system 1 can prevent the interference of the operation from the technician who is not involved with the imaging about the operation of the patient information during the imaging (mainly the imaging condition). Accordingly, in the present medical image imaging system 1, the operation of the patient information and other information during the imaging is restricted to the particular technician or tablet terminal to ensure the safe operation.

Modifications

In the present modification, the operation authority control process in the embodiment is performed in the operation authority control apparatus provided between the medical image imaging apparatus 3 and the user terminal group 5. That is, the processing circuitry in the operation authority control apparatus has the determination function 195 and the assignment function 197 illustrated in FIG. 2. The operation authority control apparatus is realized by, for example, a server apparatus that performs the operation authority control process.

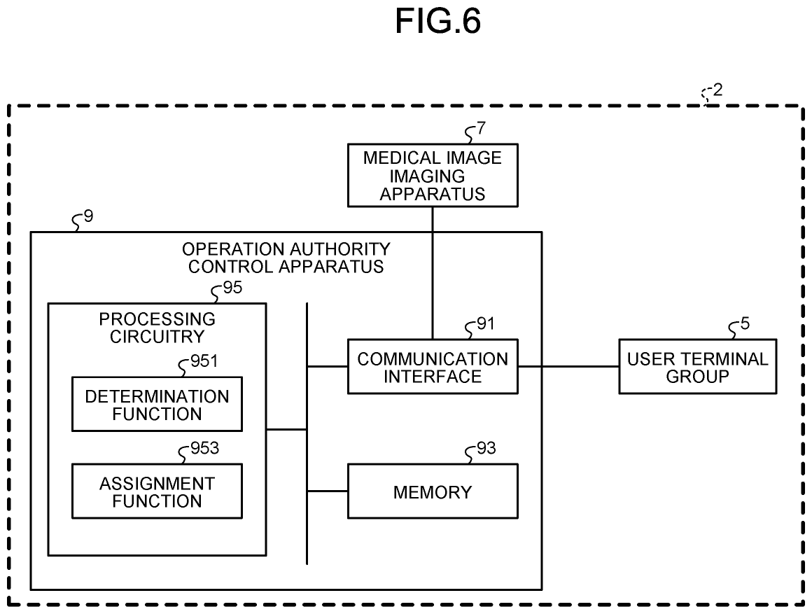

FIG. 6 is a diagram illustrating one example of a structure of a medical image imaging system 2 according to the present modification. As illustrated in FIG. 6, the medical image imaging system 2 includes a medical image imaging apparatus 7, an operation authority control apparatus 9, and the user terminal group 5. The medical image imaging apparatus 7 in the present modification corresponds to a normal modality that does not have the determination function 195 or the assignment function 197. The operation authority control apparatus 9 includes a communication interface 91, a memory 93, and a processing circuitry 95. The processing circuitry 95 has a determination function 951 and an assignment function 953. The communication interface 91, the memory 93, and the processing circuitry 95 in the present modification have various functions related to the operation authority control process. For this reason, the description about the determination function 951, the assignment function 953, the communication interface 91, and the memory 93 is omitted. In addition, the procedure and effect of the operation authority control process in the present modification are also similar to those in the embodiment; thus, the description is omitted.

### First Application Example

In a first application example of the embodiment, the operation authority for the medical image imaging apparatus 3 is automatically set to the user terminal related to a subject whose image is imaged subsequent to the subject P (hereinafter referred to as the next imaging subject) in response to the removal of the operation authority. For example, the processing circuitry 19 causes the assignment function 197 to set the operation authority to one user terminal related to the next imaging subject (hereinafter referred to as the next user terminal) in response to the removal of the operation authority from one user terminal for which the operation authority has been set.

To make the description more specific, the next user terminal is described as the second user terminal 53 related to the second technician E2. In addition, description is made of the case in which the progress in the imaging workflow is determined to be the pre-imaging stage with respect to the second user terminal 53 before the progress in the imaging workflow is determined to be the post-imaging stage with respect to the tablet terminal 200 corresponding to the first user terminal 51 to which the operation authority has been assigned. In these cases, the assignment function 197 assigns the operation authority to the second user terminal 53 in response to the removal of the operation authority from the tablet terminal 200 (first user terminal 51). This allows the second technician to perform various operations on the MRI apparatus 4.

In the medical image imaging system 1 according to the present application example, the operation authority is assigned to one user terminal related to the subject whose image is imaged subsequent to the subject P in response to the removal of the operation authority from one user terminal. Thus, the medical image imaging system 1 according to the present application example can improve the throughput of examinations related to the medical imaging because the operation authority is assigned as the operation authority is removed.

### Second Application Example

In the present application example, in the case where a plurality of imaging conditions related to the next imaging subject are input by the user terminals, the imaging conditions are stored and in the case where the operation authority is set to the next user terminal, the imaging condition selected from among the imaging conditions in accordance with the instruction from the next user terminal is set as the imaging condition related to the next imaging subject. The next user terminal in the present application example is described below as the third user terminal 55.

When the imaging conditions related to the subject whose image is to be imaged subsequent to the subject P are input by the user terminals, the memory 13 stores therein the imaging conditions in association with the patient information of the next imaging subject. In other words, when the imaging conditions related to the same subject are input by the user terminals, the memory 13 stores the imaging conditions therein without overwriting the imaging condition.

The processing circuitry 19 causes the assignment function 197 to assign the operation authority to the third user terminal 55 when the progress in the imaging workflow is determined to be the pre-imaging stage in the third user terminal 55. The assignment function 197 assigns the selected imaging condition among the imaging conditions as the imaging condition related to the next imaging subject in accordance with the instruction from the third user terminal 55 by the third technician E3.

In the medical image imaging system 1 according to the present application example, in the case where the imaging conditions related to the subject whose image is imaged subsequent to the subject P are input by the user terminals, the imaging conditions are stored, and in the case where the operation authority is assigned to one user terminal related to the subject whose image is imaged subsequent to the subject P, the imaging condition selected by the user among the imaging conditions in accordance with the instruction from the one user terminal related to the subject whose image is imaged subsequent to the subject P is assigned as the imaging condition related to the subject whose image is imaged subsequent to the subject P.

As a result, in the medical image imaging system 1 according to the present application example, the imaging conditions related to the next imaging subject are stored temporarily in the memory 13 without being overwritten; therefore, the user related to the next imaging subject can select and set the optimal imaging condition from among the imaging conditions. This allows the medical image imaging system 1 according to the present application example to improve the throughput of the imaging related to the next imaging subject.

### Third Application Example

In the present application example, the operation authority assigned by the assignment function 197 is automatically removed according to a predetermined condition. In other words, when the predetermined condition is satisfied, the console apparatus 33 collects the operation authority from the tablet terminal 200 to which the operation authority has been assigned. To make the description more specific, the tablet terminal 200 to which the operation authority has been assigned is described as the first user terminal 51.

The predetermined condition is that, for example, the operation is not input for a predetermined time in the first user terminal 51 (hereinafter referred to as operation non-input state), and in the case where the first technician E1 operates the first user terminal 51, the request for removing the operation authority (hereinafter referred to as authority removal request) is transmitted from the console apparatus 33 to the first user terminal 51 and the approval for the removal of the operation authority by the first technician E1 (hereinafter referred to as approval for authority removal) is received in the first user terminal 51.

The operation non-input state corresponds, for example, to a state in which communication between the first user terminal 51 and the MRI apparatus 4 stops for a predetermined time. The predetermined time is a few minutes, for example, one minute. The authority removal request is generated, for example, in response to a system error in the medical image imaging apparatus 3 such as the MRI apparatus 4, a patient call by the subject P, or an urgent event such as an outpatient visit requiring urgency.

In the case where the operation is input for a predetermined time in one user terminal for which the operation authority has been set and the event related to the removal of the operation authority occurs in the medical image imaging apparatus 3, the communication interface 11 transmits the request for removing the operation authority to the one user terminal for which the operation authority has been set. At this time, the first user terminal 51 displays the authority removal request. When the first technician E1 inputs the approval for the authority removal in the first user terminal 51, the first user terminal 51 transmits a signal related to the approval for the authority removal (hereinafter referred to as an approval signal) to the console apparatus 33. The communication interface 11 outputs the approval signal to the processing circuitry 19.

In the case where the operation is not input for a predetermined time in one user terminal (first user terminal 51) for which the operation authority has been set, the processing circuitry 19 causes the assignment function 197 to remove the operation authority from the first user terminal 51 to which the operation authority has been assigned. The assignment function 197 removes the operation authority from the first user terminal 51 in response to the reception of the approval for the authority removal request, i.e., the reception of the approval signal from the first user terminal 51.

In the medical image imaging system 1 according to the present application example, if the operation is not input for a predetermined time in one user terminal to which the operation authority has been assigned, the operation authority is removed from the one user terminal. As a result, in this medical image imaging system 1, when the communication between the user terminal for which the operation authority has been set and the medical image imaging apparatus 3 stops for a predetermined time, the operation authority can be removed from the user terminal to which the operation authority has been assigned. Therefore, this medical image imaging system 1 can prevent the user terminal to which the operation authority has been assigned from being isolated.

In the present medical image imaging system 1 according to the present application example, in the case where the operation is input for a predetermined time in one user terminal to which the operation authority has been assigned and the event related to the removal of the operation authority occurs in the medical image imaging apparatus 3, the request for removing the operation authority is transmitted to the one user terminal to which the operation authority has been assigned and in response to the reception of the approval for the request, the operation authority is removed from the one user terminal. Thus, by the present medical image imaging system 1, and in the occurrence of the urgent event, the operation authority can be removed from the user terminal to which operation authority has been assigned. Accordingly, for example, the console apparatus 33 can handle the urgent event.

As a result, the medical image imaging system 1 according to the present application example can improve the operability and the safety related to the medical image imaging apparatus 3.

In the case where the technical concept in the embodiment and the like is achieved by an operation authority control method, the operation authority control method determines the progress in the workflow related to the imaging of the subject P by the medical image imaging apparatus 3, and assigns the operation authority for the medical image imaging apparatus 3 to one user terminal among the user terminals on the basis of the determined progress, and in the case where the timing after the imaging of the subject is determined to be the progress in the workflow, removes the operation authority assigned to the one user terminal. The procedure and effect of the operation authority control process by the operation authority control method are similar to those in the embodiment and the like; therefore, the description is omitted.

In the case where the technical concept in the embodiment and the like is achieved by an operation authority control program, the operation authority control program causes a computer to determine the progress in the workflow related to the imaging of the subject P by the medical image imaging apparatus 3, and assign the operation authority for the medical image imaging apparatus 3 to one user terminal among the user terminals on the basis of the determined progress. The procedure and effect of the operation authority control process in the operation authority control program are similar to those in the embodiment and the like, and therefore the description is omitted.

According to at least one of the above-described embodiments, etc., the safety of the operation of the modality by multiple users can be improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image imaging system comprising a processing circuitry configured to:
determine a progress in a workflow related to imaging of a subject by a medical image imaging apparatus; and
assign an operation authority for the medical image imaging apparatus to one user terminal among a plurality of user terminals in accordance with the determined progress.

2. The medical image imaging system according to claim 1, wherein in a case where the processing circuitry determines a timing after the imaging of the subject to be the progress in the workflow, the processing circuitry is configured to remove the operation authority assigned to the one user terminal.

3. The medical image imaging system according to claim 1, wherein the progress related to the assignment of the operation authority is at least one of a room entry guidance timing when the subject is guided into an examination room where the medical image imaging apparatus is disposed, a placement timing when the subject is placed on a couchtop of a couch in the medical image imaging apparatus, a movement timing when the couchtop is moved to an opening of a gantry including an image imaging system in the medical image imaging apparatus, and a setting timing when an imaging condition related to the imaging is set in the examination room.

4. The medical image imaging system according to claim 3, wherein the room entry guidance timing is at least one of a timing when the one user terminal approaches within a range of a predetermined distance from the examination room, a timing when the one user terminal is placed at a predetermined place before the examination room, and a timing when the subject or a user who operates the one user terminal is authenticated about entry into the examination room.

5. The medical image imaging system according to claim 3, wherein the placement timing is at least one of a timing when the one user terminal approaches within a range of a first distance from the couch, a timing when the one user terminal is placed at a predetermined place in the examination room, a timing when the subject or a user who operates the one user terminal is authenticated within a range of a second distance from the couch or the gantry, and a timing when the user touches an operation panel for movement of the couch or the couchtop.

6. The medical image imaging system according to claim 3, wherein the movement timing is at least one of a timing when the user inputs an operation of the couchtop on an operation panel for movement of the couchtop, a timing when movement control of the couchtop is input by the one user terminal, and a timing when the subject placed on the couchtop is specified based on output from an optical camera.

7. The medical image imaging system according to claim 3, wherein the setting timing is at least one of a timing when the imaging condition is set by the one user terminal in the examination room, a timing when the imaging condition is set by the one user terminal in a state where the subject is moved to a center of the imaging in the opening, and a timing when the user exits from the examination room in the state where the subject is moved to the center of the imaging.

8. The medical image imaging system according to claim 2, wherein the timing after the imaging of the subject as the progress in the workflow is a room exit guidance timing when the subject is guided from the examination room where the medical image imaging apparatus is disposed, to outside of the examination room.

9. The medical image imaging system according to claim 8, wherein the room exit guidance timing is at least one of a timing when the couchtop of the couch in the medical image imaging apparatus is separated from the center of the imaging of the opening of the gantry including the image imaging system in the medical image imaging apparatus, a timing when the couchtop descends toward a floor surface of the examination room, a timing when, in a case where the medical image imaging apparatus is a magnetic resonance imaging apparatus, a reception coil is detached from a coil port at the couchtop, a timing when separation of the subject from the couchtop is specified based on output from an optical camera, a timing when the one user terminal has reached a third distance from the gantry or the couch, and a timing when end of the imaging is input in the one user terminal.

10. The medical image imaging system according to claim 2, wherein the processing circuitry is configured to assign the operation authority to one user terminal related to a subject whose image is imaged subsequent to the subject in response to removal of the operation authority from the one user terminal.

11. The medical image imaging system according to claim 1, further comprising a memory configured to, in a case where a plurality of imaging conditions related to a subject whose image is imaged subsequent to the subject are input by the user terminals, store the imaging conditions therein, wherein in a case where the operation authority is assigned to one user terminal related to the subject whose image is imaged subsequent to the subject, the processing circuitry is configured to assign the imaging condition selected by a user among the imaging conditions as the imaging related to the subject whose image is imaged subsequent to the subject, based on an instruction from the one user terminal related to the subject whose image is imaged subsequent to the subject.

12. The medical image imaging system according to claim 1, wherein in a case where an operation is not input for a predetermined time in the one user terminal to which the operation authority is assigned, the processing circuitry is configured to remove the operation authority from the one user terminal.

13. The medical image imaging system according to claim 1, further comprising a communication interface configured to, in a case where an operation is input for a predetermined time in the one user terminal to which the operation authority is assigned and an event related to removal of the operation authority occurs in the medical image imaging apparatus, transmit a request for removing the operation authority to the one user terminal to which the operation authority is assigned, wherein the processing circuitry is configured to remove the operation authority from the one user terminal in response to reception of approval for the request.

14. The medical image imaging system according to claim 1, wherein an operation related to the operation authority is at least one of an operation of patient information related to the subject during the imaging by the medical image imaging apparatus, an operation of the patient information related to the subject whose image is imaged by the medical image imaging apparatus, an operation related to start, pause, and stop of the imaging by the medical image imaging apparatus, and an operation related to control of the couch in the medical image imaging apparatus.

15. The medical image imaging system according to claim 14, wherein the patient information includes an imaging condition.

16. An operation authority control method comprising:
determining a progress in a workflow related to imaging of a subject by a medical image imaging apparatus;
assigning an operation authority for the medical image imaging apparatus to one user terminal among a plurality of user terminals in accordance with the determined progress; and
in a case where a timing after the imaging of the subject is determined to be the progress in the workflow, removing the operation authority assigned to the one user terminal.

17. A non-transitory computer-readable nonvolatile storage medium storing therein an operation authority control program that causes a computer to execute:
determining a progress in a workflow related to imaging of a subject by a medical image imaging apparatus; and
assigning an operation authority for the medical image imaging apparatus to one user terminal among a plurality of user terminals in accordance with the determined progress.
